(19) United States
(12) Patent Application Publication
(10) Pub. No.: US 2025/0167899 A1
(43) Pub. Date: May 22, 2025

(54) MEASURING ATMOSPHERIC GREENHOUSE GAS CONCENTRATIONS USING TELECOMMUNICATION NETWORK INFRASTRUCTURE

(71) Applicant: T-MOBILE INNOVATIONS LLC, Overland Park, KS (US)

(72) Inventors: Lyle Walter PACZKOWSKI, Mission Hills, KS (US); Galip Murat KARABULUT, Vienna, VA (US)

(21) Appl. No.: 18/513,274

(22) Filed: Nov. 17, 2023

(51) Int. Cl. H04B 17/318; G06F 9/54; G08B 21/12

(52) U.S. Cl. CPC H04B 17/318; G06F 9/541; G08B 21/12

(57) ABSTRACT

The present technology relates to a systems and methods for monitoring gas concentrations, specifically greenhouse gasses, using a cellular network. A target site receives a signal with predetermined signal strength from a base station. Changes in signal strength are monitored to determine alterations in gas concentration, providing near real-time access to this data through an application programing interface. The technology also provides for monitoring multiple base stations, aggregating data, and creating a near real-time geographical map of greenhouse gas concentrations.

### TECHNICAL FIELD

The present invention relates to the field of wireless telecommunications and networks, specifically to detecting gas concentrations using the wireless telecommunications and networks.

### SUMMARY

The present disclosure describes a method and system for monitoring gas concentrations utilizing a cellular network. A target site receives a signal with predetermined signal strength from a base station. Changes in the signal are monitored to determine changes in greenhouse gas concentrations.

### BRIEF DESCRIPTION OF THE DRAWINGS

The present technology is described in detail herein with reference to the drawing figures, which are intended to be exemplary and non-limiting in nature, wherein:

FIG. 1 depicts an exemplary computing environment suitable for use in implementation of the present disclosure;

FIG. 2 illustrates a diagram of an exemplary network environment in which implementations of the present disclosure may be employed;

FIG. 3 illustrates a diagram of an exemplary network environment in which implementations of the present disclosure may be employed;

FIG. 4 illustrates a diagram of an exemplary network environment in which implementations of the present disclosure may be employed;

FIG. 5 illustrates a diagram of an exemplary network environment in which implementations of the present disclosure may be employed;

FIG. 6 illustrates a diagram of an exemplary network environment in which implementations of the present disclosure may be employed;

FIG. 7 depicts a block diagram of an exemplary method, in accordance with an embodiment of the present technology; and

FIG. 8 depicts a block diagram of an exemplary method, in accordance with an embodiment of the present technology.

### DETAILED DESCRIPTION

The subject matter of embodiments of the invention is described with specificity herein to meet statutory requirements. However, the description itself is not intended to limit the scope of this patent. Rather, the inventors have contemplated that the claimed subject matter might be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

With the rise of industrialization and urbanization, concerns about environmental degradation, particularly increased levels of greenhouse gasses in the atmosphere, have surged. Traditional methods of monitoring greenhouse gasses concentrations are limited, often offering coverage constrained to specific geographic areas and times, making it imperative to explore more comprehensive monitoring solutions. Existing greenhouse gas sensors such as electrochemical, non-dispersive infrared (NDIR), and metal oxide semiconductor (MOS) sensors have inherent challenges; they are expensive, have limited sensitivity to low concentration levels, and a substantial lag in data reporting. These sensors are typically dedicated devices deployed in a static location providing data from fixed points that may not effectively represent broader areas. Consequently, there are significant data gaps and latency in the existing frameworks of environmental monitoring. These delays can impede timely decision-making and responses to acute environmental crises, underscoring the pressing need for real-time monitoring systems.

Additionally, the current systems necessitate regular maintenance and calibration to maintain accuracy, a process that is both time-consuming and resource-intensive. The spatial resolution of existing technologies is often inadequate, failing to detect variations over smaller geographical areas and sometimes providing incomplete pictures of the greenhouse gas concentration landscape. The associated high installation and operational costs further emphasize the demand for a more cost-effective and energy-efficient solution.

In light of these challenges, the emergence of large scale cellular networks presents untapped potential to revolutionize environmental monitoring. These networks, with their existing extensive coverage and advanced infrastructure, can be harnessed to monitor greenhouse gasses concentrations. This technology facilitates the detection of greenhouse gasses concentrations by analyzing changes in signal strength caused by varying greenhouse gas concentrations in the air interface can double as a vast environmental sensor grid.

In one aspect, the present invention involves a system comprising a target site, which is equipped with one or more processors and computer storage hardware devices. These devices store computer-usable instructions that, when executed, enable the processors to receive a radio frequency (RF) signal transmitted from a base station, with this signal possessing a predetermined signal strength. The system monitors any variations in the detected signal strength of this signal. Further, the system can ascertain changes in gas concentration by interpreting the monitored alterations in signal strength, wherein each unit of signal strength change corresponds to a predetermined alteration in gas concentration. Furthermore, upon determining any changes in gas concentration, the system provides near real-time access to this information through an API.

In an additional aspect, a method is provided which uses a cellular network for the detection of atmospheric greenhouse gasses. The method encompasses receiving an RF signal at a target, the signal being transmitted from a first base station with a predetermined signal strength. The process involves continuous monitoring of the received signal strength of the RF signal to identify any deviations from the predetermined signal strength. Subsequently, changes in the concentration of greenhouse gasses are determined based on the observed alterations in the received signal strength, with each unit change in signal strength corresponding to a predetermined change in greenhouse gasses concentration. Once a change in greenhouse gasses concentration is ascertained, the method ensures that access to this change in concentration is provided through an API in near real-time.

In another aspect, a method is provided involving computer-readable media equipped with instructions for monitoring a network of base stations, each transmitting an RF signal to distinct target sites using a predetermined signal strength. The central processing unit receives data indicative of changes in signal strength at each target site and determines alterations in greenhouse gasses concentration corresponding to these changes. Subsequently, the unit aggregates these data to generate a representation of varied greenhouse gasses concentrations across the geographical region covered by the base stations and target sites. A near real-time map visualizing the distribution of greenhouse gasses concentrations is created and transmitted to a data repository. This repository is accessible by authorized users through a secure network interface.

Throughout this disclosure, several acronyms and shorthand notations are employed to aid the understanding of certain concepts pertaining to the associated system and services. These acronyms and shorthand notations are intended to help provide an easy methodology of communicating the ideas expressed herein and are not meant to limit the scope of embodiments described in the present disclosure. The following is a list of these acronyms.
3G Third-Generation Wireless Technology
4G Fourth-Generation Cellular Communication System
CD-ROM Compact Disk Read Only Memory
CDMA Code Division Multiple Access
eNodeB Evolved Node B
GIS Geographic/Geographical/Geospatial Information System
GPRS General Packet Radio Service
GSM Global System for Mobile communications
iDEN Integrated Digital Enhanced Network
DVD Digital Versatile Discs
EEPROM Electrically Erasable Programmable Read Only Memory
LED Light Emitting Diode
LTE Long Term Evolution
MD Mobile Device
PC Personal Computer
PCS Personal Communications Service
PDA Personal Digital Assistant
RAM Random Access Memory
RET Remote Electrical Tilt
RF Radio-Frequency
RFI Radio-Frequency Interference
R/N Relay Node
RNR Reverse Noise Rise
ROM Read Only Memory
RSRP Reference Transmission Receive Power
RSRQ Reference Transmission Receive Quality
RSSI Received Transmission Strength Indicator
SINR Transmission-to-Interference-Plus-Noise Ratio
SNR Transmission-to-noise ratio
SON Self-Organizing Networks
TDMA Time Division Multiple Access
UMTS Universal Mobile Telecommunications Systems

Further, various technical terms are used throughout this description. An illustrative resource that describes these terms may be found in Newton's Telecom Dictionary, 32nd Edition (2022).

A “mobile device,” as used herein, is a device that has the capability of using a wireless communications network, and may also be referred to as a “user device,” “wireless communication device,” or “user equipment (UE).” A mobile device may take on a variety of forms, such as a personal computer (PC), a laptop computer, a tablet, a mobile phone, a personal digital assistant (PDA), a server, or any other device that is capable of communicating with other devices using a wireless communications network. Additionally, embodiments of the present technology may be used with different technologies or standards, including, but not limited to, CDMA 1×A, GPRS, EvDO, TDMA, GSM, WiMax technology, LTE, and/or LTE Advanced, among other technologies and standards.

Embodiments of the technology may be embodied as, among other things, a method, a system, and/or a computer-program product. Accordingly, the embodiments may take the form of a hardware embodiment, or an embodiment combining software and hardware. In one embodiment, the technology may take the form of a computer-program product that includes computer-useable instructions embodied on one or more computer-readable media.

Computer-readable media may include both volatile media, non-volatile media, removable media, non-removable media, and contemplate media readable by a database, a switch, and/or various other network devices. Network switches, routers, and related components are conventional in nature, as are methods of communicating with the same. By way of example, and not limitation, computer-readable media may include computer storage media and/or communications media.

Computer storage media, or machine-readable media, may include media implemented in any method or technology for storing information. Examples of stored information include computer-useable instructions, data structures, program modules, and other data representations. Computer storage media may include, but are not limited to, RAM, ROM, EEPROM, flash memory or other memory technology, CD-ROM, digital versatile discs (DVD), holographic media or other optical disc storage, magnetic cassettes, magnetic tape, magnetic disk storage, and other storage devices. These memory components may store data momentarily, temporarily, and/or permanently.

Communications media typically store computer-useable instructions-including data structures and program modules-in a modulated data signal. The term “modulated data signal” refers to a propagated signal that has one or more of its characteristics set or changed to encode information in the signal. Communications media includes any information-delivery media. By way of example, but not limitation, communications media may include wired media, such as a wired network or direct-wired connection, and wireless media such as acoustic, infrared, radio, microwave, spread-spectrum, and other wireless media technologies. Combinations of the above are included within the scope of computer-readable media. Communications media do not include signals per se.

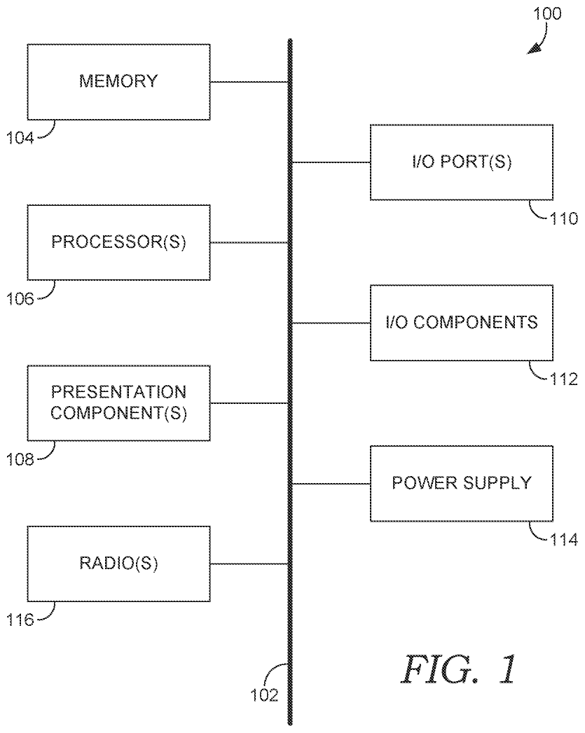

Referring to the drawings in general, and initially to FIG. 1, an exemplary computing environment 100 suitable for practicing embodiments of the present technology is provided. Computing environment 100 is but one example, and is not intended to suggest any limitation as to the scope of use or functionality of the embodiments discussed herein. Neither should the computing environment 100 be interpreted as having any dependency or requirement relating to any one or a combination of components illustrated. It should be noted that although some components in FIG. 1 are shown in the singular, they may be plural. For example, the computing environment 100 might include multiple processors and/or multiple radios. As shown in FIG. 1, computing environment 100 includes a bus that directly or indirectly couples various components together, including memory 104, processor(s) 106, presentation component(s) 108 (if applicable), radio(s) 116, input/output (I/O) port(s) 110, input/output (I/O) component(s) 112, and power supply 114. More or fewer components are possible and contemplated, including in consolidated or distributed form.

Memory 104 may take the form of memory components described herein. Thus, further elaboration will not be provided here, but it should be noted that memory 104 may include any type of tangible medium that is capable of storing information, such as a database. A database may be any collection of records, data, and/or information. In one embodiment, memory 104 may include a set of embodied computer-executable instructions that, when executed, facilitate various functions or elements disclosed herein. These embodied instructions will variously be referred to as “instructions” or an “application” for short. Processor 16 may actually be multiple processors that receive instructions and process them accordingly. Presentation component 108 may include a display, a speaker, and/or other components that may present information (e.g., a display, a screen, a lamp (LED), a graphical user interface (GUI), and/or even lighted keyboards) through visual, auditory, and/or other tactile cues.

Radio 116 may facilitate communication with a network, and may additionally or alternatively facilitate other types of wireless communications, such as Wi-Fi, WiMAX, LTE, and/or other VoIP communications. In various embodiments, the radio 20 may be configured to support multiple technologies, and/or multiple radios may be configured and utilized to support multiple technologies.

The input/output (I/O) ports 110 may take a variety of forms. Exemplary I/O ports may include a USB jack, a stereo jack, an infrared port, a firewire port, other proprietary communications ports, and the like. Input/output (I/O) components 112 may comprise keyboards, microphones, speakers, touchscreens, and/or any other item usable to directly or indirectly input data into the computing environment 100.

Power supply 114 may include batteries, fuel cells, and/or any other component that may act as a power source to supply power to the computing environment 10 or to other network components, including through one or more electrical connections or couplings. Power supply 26 may be configured to selectively supply power to different components independently and/or concurrently.

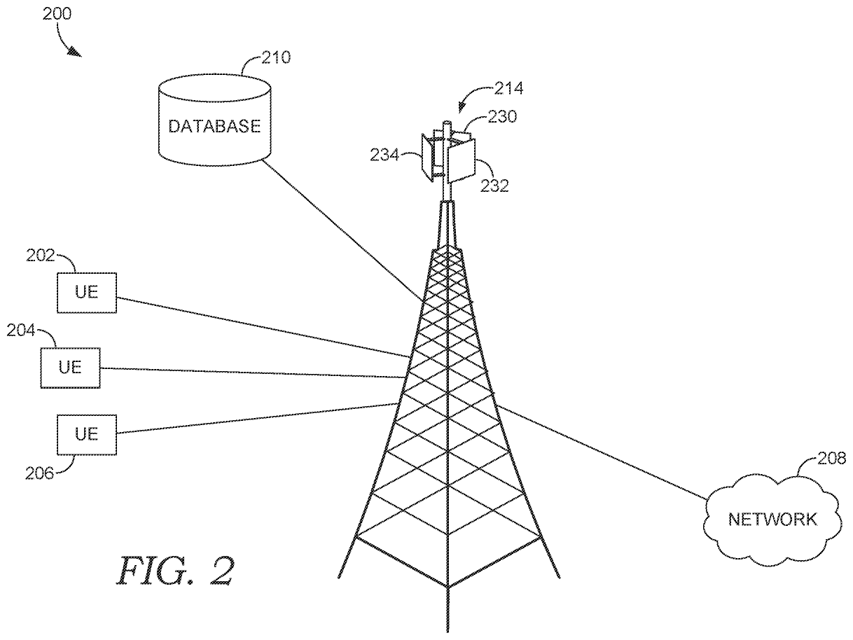

FIG. 2 provides an exemplary network environment in which implementations of the present disclosure may be employed. Such a network environment is illustrated and designated generally as network environment 200. Network environment 200 is but one example of a suitable network environment and is not intended to suggest any limitation as to the scope of use or functionality of the invention. Neither should the network environment be interpreted as having any dependency or requirement relating to any one or combination of components illustrated.

Network environment 200 includes one or more user devices (e.g., user devices 202, 204, and 206), cell site 214, network 208, and database 210. In network environment 200, user devices may take on a variety of forms, such as a personal computer (PC), a user device, a smart phone, a smart watch, a laptop computer, a mobile phone, a mobile device, a tablet computer, a wearable computer, a personal digital assistant (PDA), a server, a CD player, an MP3 player, a global positioning system (GPS) device, a video player, a handheld communications device, a workstation, a router, an access point, and any combination of these delineated devices, or any other device that communicates via wireless communications with a cell site 214 in order to interact with a public or private network.

In some aspects, the user devices 202, 204, and 206 correspond to computing environment 100 in FIG. 1. Thus, a user device may include, for example, a display(s), a power source(s) (e.g., a battery), a data store(s), a speaker(s), memory, a buffer(s), a radio(s) and the like. In some implementations, the user devices 202, 204, and 206 comprises a wireless or mobile device with which a wireless telecommunication network(s) may be utilized for communication (e.g., voice and/or data communication). In this regard, the user device may be any mobile computing device that communicates by way of a wireless network, for example, a 3G, 4G, 5G, LTE, CDMA, or any other type of network.

In some cases, the user devices 202, 204, and 206 in network environment 200 may optionally utilize network 208 to communicate with other computing devices (e.g., a mobile device(s), a server(s), a personal computer(s), etc.) through cell site 214. The network 208 may be a telecommunications network(s), or a portion thereof. A telecommunications network might include an array of devices or components (e.g., one or more base stations), some of which are not shown. Those devices or components may form network environments similar to what is shown in FIG. 2, and may also perform methods in accordance with the present disclosure. Components such as terminals, links, and nodes (as well as other components) may provide connectivity in various implementations. Network 208 may include multiple networks, as well as being a network of networks, but is shown in more simple form so as to not obscure other aspects of the present disclosure.

Network 208 may be part of a telecommunication network that connects subscribers to their service provider. In aspects, the service provider may be a telecommunications service provider, an internet service provider, or any other similar service provider that provides at least one of voice telecommunications and data services to any or all of the user devices 202, 204, and 206. For example, network 208 may be associated with a telecommunications provider that provides services (e.g., LTE) to the user devices 202, 204, and 206. Additionally or alternatively, network 208 may provide voice, SMS, and/or data services to user devices or corresponding users that are registered or subscribed to utilize the services provided by a telecommunications provider. Network 208 may comprise any communication network providing voice, SMS, and/or data service(s), using any one or more communication protocols, such as a 1× circuit voice, a 3G network (e.g., CDMA, CDMA2000, WCDMA, GSM, UMTS), a 4G network (WiMAX, LTE, HSDPA), or a 5G network. The network 208 may also be, in whole or in part, or have characteristics of, a self-optimizing network.

In some implementations, cell site 214 is configured to communicate with the user devices 202, 204, and 206 that are located within the geographical area defined by a transmission range and/or receiving range of the radio antennas of cell site 214. The geographical area may be referred to as the “coverage area” of the cell site or simply the “cell,” as used interchangeably hereinafter. Cell site 214 may include one or more base stations, base transmitter stations, radios, antennas, antenna arrays, power amplifiers, transmitters/receivers, digital signal processors, control electronics, GPS equipment, and the like. In particular, cell site 214 may be configured to wirelessly communicate with devices within a defined and limited geographical area. For the purposes of the present disclosure, it may be assumed that it is undesirable and unintended by the network 208 that the cell site 214 provide wireless connectivity to the user devices 202, 204, and 206 when the uses devices 202, 204, and 206 are geographically situated outside of the cell associated with cell site 214. In some aspects, user devices 202, 204, and 206 may serve as nodes in a network dedicated to environmental monitoring. In some aspects, user devices 202, 204, and 206 can be any antennas or receivers configured to receive a signal or communication from the cell site 214.

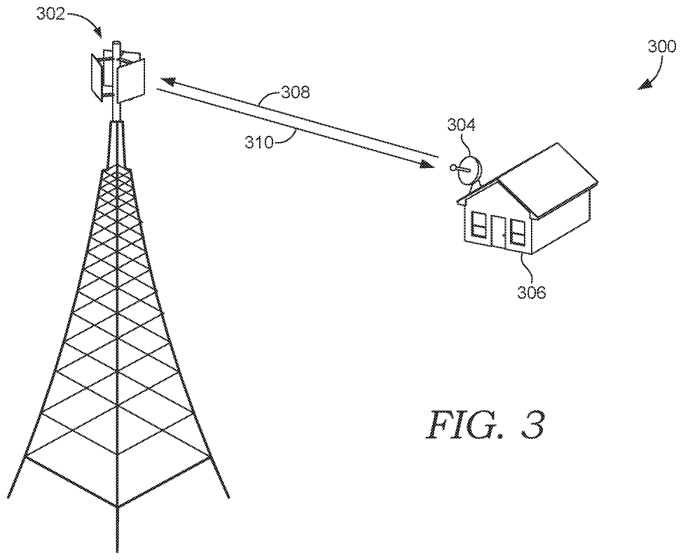

Referring to FIG. 3, an illustrative depiction of a network environment 300 is provided. Within the network environment 300, a cell site 302 (having any one or more characteristics of cell site 214 of FIG. 2) can emit a downlink signal 310 to a target receiver 304 and the target receiver 304 may communicate an uplink single 308 to the cell site 302. In embodiments described herein, the downlink signal 310 can be generated or transmitted in a plurality of frequency spectrums. In one embodiment, the downlink signal 310 operates specifically in the frequency spectrum between 30 GHz and 300 GHz. This allows for high-resolution imaging and effective penetration through certain atmospheric conditions. In other embodiments, the downlink signal 310 operates in a frequency between 1 MHz and 3 THz, offering flexibility to choose specific bands for detecting various gases and environmental parameters. In further embodiments, the downlink signal 310 operates in a frequency spectrum above 3 THz, interacting more directly with molecular vibrations and providing enhanced sensitivity. In an additional embodiment, the downlink signal 310 operates using a 47 GHz band. In other embodiments, the downlink signal 310 operates in a frequency between 26 GHz to 430 THz or 90 GHz to 140 GHz.

In some aspects, the downlink signal 310 may be a plurality of protocol data units (PDUs) encoded according to a cellular telecom protocol. In other aspects, the downlink signal may be a non-PDU signal. In some aspects, a PDU signal refers to the encapsulated data at various layers of the open systems interconnection (OSI) model, where each layer has its specific PDU format containing the data and the respective layer's header information. A PDU signal involves the transmission of these encapsulated data units over the network, carrying both user data and control information necessary for communication, while a non-PDU signal typically refers to signals that do not carry such encapsulated user or control data, possibly involved in other network functions or pure signaling purposes without carrying end-user data. At a first time the downlink signal 310 may comprise a non-PDU signal and at a second time the downlink signal 310 may be a plurality of PDUs. In some aspects, the target receiver may be configured to receive both signals comprising a plurality of PDUs and non-PDU signals. In this aspect, the non-PDU signals may be used to measure greenhouse gas concentrations at a particular time interval. For example, at a first time, the downlink signal 310 may comprise a plurality of PDUs which is not being measured for deviations to measure greenhouse gas concentrations. At a second predetermined time, the downlink signal 310 may be a non-PDU signal and may be measured for deviations to measure the greenhouse gas concentrations along the radio link path.

In aspects herein, a phased array may be used to beamform the downlink signal 310. By employing dynamic beamforming techniques such as digital beamforming, adaptive beamforming, hybrid beamforming, switched beamforming, null steering, feedback-based beamforming, or other techniques, the transceiver shapes and steers the columnar beam with a high degree of precision, ensuring the downlink signal 310 has optimal concentration and directionality toward the target receiver 304. In one aspect, the target receiver 304 can receive and analyze the downlink signal 310. The target receiver 304 monitors the signal strength and characteristics of the downlink signal 310 to identify any anomalies or deviations from an expected value. In additional aspects, the target receiver is associated with or powered by an external power supply.

To detect deviations in the downlink signal 310, the target receiver 304 can utilize signal processing algorithms that continuously monitor the characteristics of the received downlink signal 310. These algorithms analyze the signal's amplitude, phase, frequency, and modulation, comparing them against the expected baseline characteristics and signal levels established during the calibration phase. Any variance beyond these expected values is determined to be a deviation. The system can correlate specific levels of signal attenuation or deviation to corresponding concentrations of gases by referencing pre-established look-up tables generated during calibration tests with controlled gas concentrations.

The calibration and identification of expected signal strengths, waveforms, and signal characteristics is needed such that the system is able to identify deviations that are indicative of a change in greenhouse gas concentrations. A calibration procedure can involve transmitting a series of RF signals from the cell site 302, across varying power levels, to establish a reference, baseline, or expected signal strength at the target receiver 304. During calibration, the system transmits a series of signals with varying characteristics such as power levels, frequencies, and modulations. The target receiver 304 captures these signals, and their strengths and characteristics are measured and recorded to formulate the baseline data. This data defines the expected signal characteristics and values under normal atmospheric conditions and standard gas concentrations.

The target receiver 304 can identify deviations in the intensity or modulation of the expected strength of the downlink signal, thus, the target receiver 304 can determine a correlated shift in atmospheric gas concentrations. To establish a comprehensive mapping that links specific signal strength changes to particular gas concentrations, controlled gas concentrations are introduced into the downlink pathway during tests. The target receiver 304 determines signal deviations from expected signal strengths as a function of the controlled introduction of gas concentrations. As such, the target receiver 304 is able to determine a particular change in signal equates to a particular change in concentration of gas. This data is then stored within the target receiver's 304 memory as a lookup table, or in another database such as database 210. This enables the target receiver 304 to determine specific gas concentrations by analyzing deviations in the downlink signal 310 and correlating them to a change in gas concentration. In other aspects, the target receiver can be baselined using other instrumentation or methods. For example, a secondary device may be used to measure the greenhouse gas concentration along the radio link path such as to provide calibration data.

A variety of environmental constituents can be monitored by the target receiver 304. Data sets can be compiled correlating different gas concentrations or particulate concentrations in the air versus changes in expected signal strengths. The target receiver 304 is able to detect variations in the concentrations of greenhouse gases such as carbon dioxide, water vapor, methane (CH4), nitrous oxide (N2O), and ozone (O3). Moreover, the target receiver 304 is able to monitor airborne particulates, including fine and coarse particulate matter (PM2.5 and PM10), volatile organic compounds (VOCs), and aerosols.

The gas concentration associated with the received downlink signal 310 from the cell site 302 can be determined by the target receiver 304. Leveraging the lookup table or database 210, where specific variations in signal strength are meticulously mapped to corresponding gas concentrations, the system compares deviations in the downlink signal 310 to the pre-established data points in the table or database. By associating the identified deviations in the downlink signal 310 with the correlated shifts in the atmospheric gas concentrations stored in the memory, the target receiver 304 can precisely determine the present concentration or change in concentration of gas in the atmosphere.

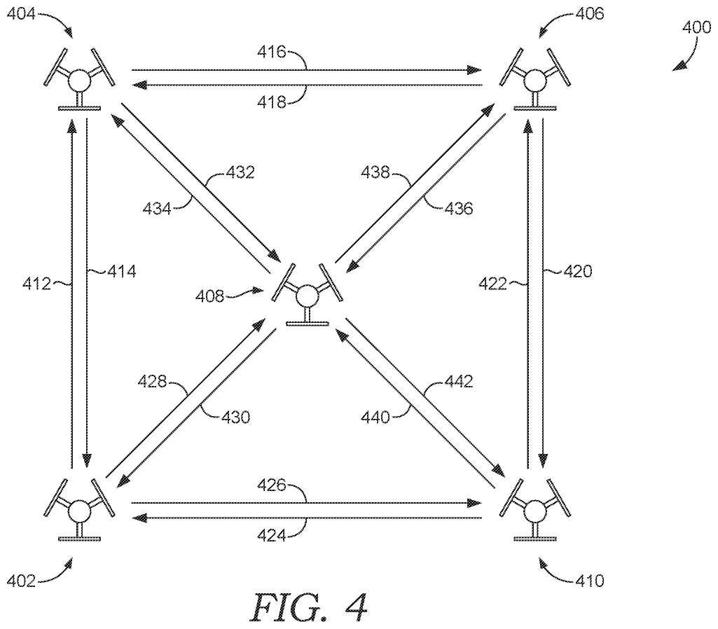

Continuing now with FIG. 4, FIG. 4 illustrates a detailed network configuration showing the interconnectedness between a series of cell sites: the first cell site 402, the second cell site 404, the third cell site 406, the fourth cell site 410, and the fifth cell site 408. Within this network environment, the first cell site 402 establishes communication with the second cell site 404 through the exchange of a first signal 414 and a second signal 412. The second cell site 404 and the third cell site 406 communicate through the third RF signal 416 and the fourth signal 418. The third cell site 406 interacts with the fourth cell site 410 through the fifth signal 420 and the sixth signal 422. Additionally, a communication link is established between the fourth cell site 410 and the first cell site 402 through the seventh signal 424 and the eighth signal 426. Integration of the Fifth Cell Site 408 can have bi-directional communication established with the first cell site 402 through the ninth signal 428 and the tenth signal 430. Similarly, interactions between the fifth cell site 408 and the second cell site 404, as well as the third cell site 406, are facilitated through the eleventh and twelfth signals 432, 434 and the thirteenth and fourteenth signals 436, 438 respectively. Further, the fourth cell site 410 and the fifth cell site 408 engage in communication through the fifteenth signal 440 and the sixteenth signal 442.

Similar to the previous description with respect to FIG. 3, the signals, which may be RF signals, in this configuration may operate in various frequency spectrums. The multi-cell site configuration depicted in FIG. 4 allows for a layered approach to environmental monitoring. The network of cell sites, through their communication web, collaboratively analyzes atmospheric conditions, identifying and quantifying variations indicative of changes in environmental factors such as gas concentrations and particulate matter at each cell site. The collected environmental data from this multi-cell site network is channeled into a user interface, providing real-time visualizations and historical data trends. The data from various cell sites allows for more accurate and comprehensive monitoring. Depending on the geographical and infrastructural layout, additional cell sites and communication links can be integrated.

Each cell site within the depicted network in FIG. 4 is equipped with receivers and transceivers that facilitate the detection of greenhouse gas concentrations in the atmosphere, as described with respect to the cell site 302 and target receiver 304 in FIG. 3. As such, each cell site in FIG. 4 is capable of transmission and receiving. When a RF signal is emitted from one cell site to another, it traverses through the air, interacting with various atmospheric components including carbon dioxide molecules. The alterations in the signal characteristics due to this interaction are monitored by the receiving cell site. Upon receiving a signal, each cell site analyzes the alterations in signal characteristics from expected signal characteristics, such as phase shifts, signal strength attenuations, and waveform modulations.

These alterations are indicative of the presence and concentration of greenhouse gasses, or other particulates, in the path. Signal processing units within each cell site interpret these alterations, converting them into tangible data regarding gas concentrations.

The data obtained from the analysis of signals at each cell site can then be compiled to construct a comprehensive map of gas concentrations across the geographic region covered by the network. Each cell site contributes localized data, which is combined to provide a detailed representation of the variations in greenhouse gas levels across different locations. Predictive algorithms within the system further utilize accumulated historical and spatial data to forecast potential future trends and fluctuations in greenhouse gas concentrations across the mapped region.

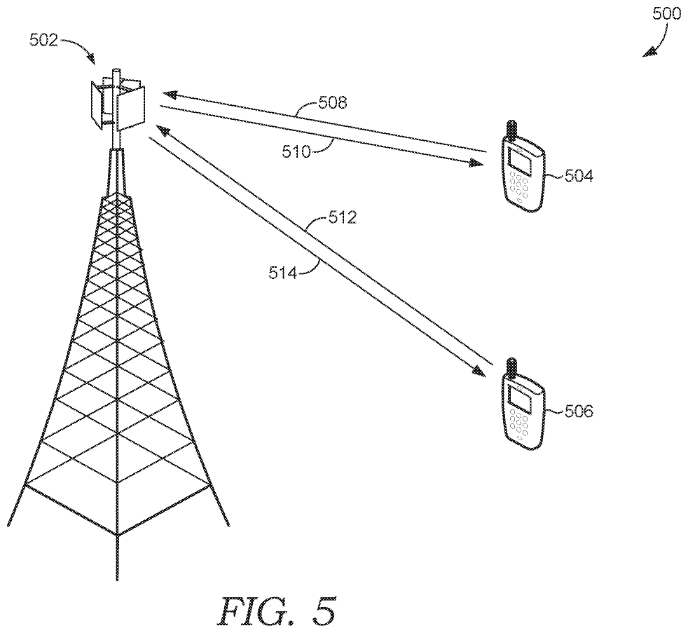

Turning to FIG. 5, this figure describes a network environment 500 that comprises a cell site 502, a first UE 504, and a second UE 506. Within this network environment 500, cell site 502 and the first UE 504 are interconnected through a bi-directional communication channel, consisting of a first downlink signal 508 and a first uplink signal 510.

The cell site 502 also forms a communication link with the second UE 506 through a second downlink signal 512 and a second uplink signal 514. Similar to the configuration depicted in FIG. 3, the downlink signals within the network environment of FIG. 5 may operate across various frequency spectrums. Downlink signals from cell site 502 to the UEs interact with atmospheric components, including carbon dioxide, causing variations in signal characteristics such as phase shifts, attenuation, and modulation. These variations, indicative of greenhouse gas or particulate matter concentration, are analyzed and interpreted by the UEs, converting them into quantifiable greenhouse gas concentration data, as described with respect to FIG. 3. The accumulated data can then be utilized to construct a comprehensive map of carbon dioxide concentrations across the covered geographic region. The integration of location-specific data for the first UE 504 and the second UE 506 along with concentration values for each UE, enables the generation of a geographic map of current concentration data. The system also incorporates predictive algorithms that leverage historical and spatial data to anticipate potential future trends and fluctuations in carbon dioxide concentrations across the region.

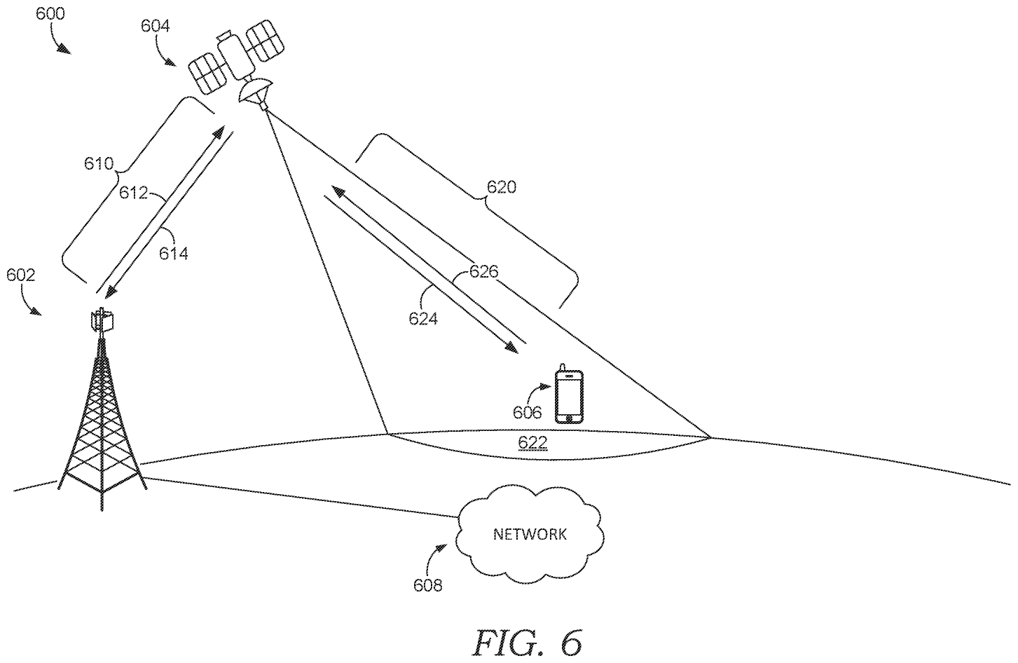

Turning now to FIG. 6, an exemplary network environment is illustrated in which implementations of the present disclosure may be employed. Such a network environment is illustrated and designated generally as network environment 600. At a high level the network environment 600 comprises a gateway 602, a satellite 604 of a satellite RAN, a UE 606, and a network 608. Satellite 604 or any other satellite may be referred to as an extraterrestrial base station herein. In some embodiments, an extraterrestrial base station refers to a satellite such as satellite 604 or space station. Though the composition of network environment 600 illustrates objects in the singular, it should be understood that more than one of each component is expressly conceived as being within the bounds of the present disclosure; for example, the network environment 600 may comprise multiple gateways, multiple distinct networks, multiple UEs, multiple satellites that communicate with a single gateway, and the like. Similarly, though certain objects of network environment 600 are illustrated in a certain form, it should be understood that they may take other forms; for example, even though the UE 606 is illustrated as a cellular phone, a UE suitable for implementations with the present disclosure may be any computing device having any one or more aspects described with respect to FIG. 1.

The network environment 600 includes a gateway 602 communicatively connected to the network 608 and the satellite 604. The gateway 602 may be connected to the network 608 via one or more wireless or wired connections and is connected to the satellite 604 via a feeder link 610. The gateway 602 may take the form of a device or a system of components configured to communicate with the UE 606 via the satellite 604 and to provide an interface between the network 608 and the satellite 604. Generally, the gateway 602 utilizes one or more antennas to transmit signals to the satellite 604 via a forward uplink 612 and to receive signals from the satellite 604 via a return downlink 614. The gateway 602 may communicate with a plurality of satellites, including the satellite 604. The network 608 comprises any one or more public or private networks, any one or more of which may be configured as a satellite network, a publicly switched telephony network (PSTN), or a cellular telecommunications network. In aspects, the network 608 may comprise a satellite network connecting a plurality of gateways (including the gateway 602) to other networks, a cellular core network (e.g., a 4G, 5G, of 6G core network, an IMS network, and the like), and a data network. In such aspects, each of the satellite network and the cellular core network may be associated with a network identifier such as a public land mobile network (PLMN), a mobile country code, a mobile network code, or the like, wherein the network identifier associated with the satellite network is the same or different than the network identifier associated with the cellular network.

The network environment 600 includes one or more satellites, represented by satellite 604. The satellite 604 is generally configured to relay communications between the gateway 602 and the UE 606. The satellite 604 communicates with the gateway using the feeder link 610 and communicates with the UE 606 using a user link 620. The user link 620 comprises a forward downlink 624 used to communicate signals from the satellite 604 to the UE 606 and a return uplink 626 used to communicate signals from the UE 606 to the satellite 604. The satellite 604 may communicate with the UE 606 using any wireless telecommunication protocol desired by a network operator, including but not limited to 3G, 4G, 5G, 6G, 802.11x and the like. Though shown as having a single RF signal providing coverage to a satellite coverage area 622, the satellite 604 may be configured to utilize a plurality of individual signals to communicate with multiple different areas at or near the same time. Similarly, though a single forward downlink 624 and a single return uplink 626 are illustrated, the UE 606 may utilize multiple downlinks and/or multiple uplinks to communicate with the satellite 604, using any one or more frequencies as desired by a satellite or network operator.

In the network environment 600, the satellite 604 functions as a base station and transmits an RF signal. This RF signal is received by the UE 606 or the gateway 602, which can be conceptualized as the target site, such as target receiver 304 of FIG. 3, equipped with processors and storage devices. The UE 606 or gateway 602 actively monitors the detected signal strength of the RF signal. Variations in the detected signal strength allow the UE 606 or gateway 602 to ascertain changes in gas concentration. As every unit of change in signal strength correlates with a predetermined change in gas concentration, real-time data about the gas concentration is relayed via an API connected to the network 608.

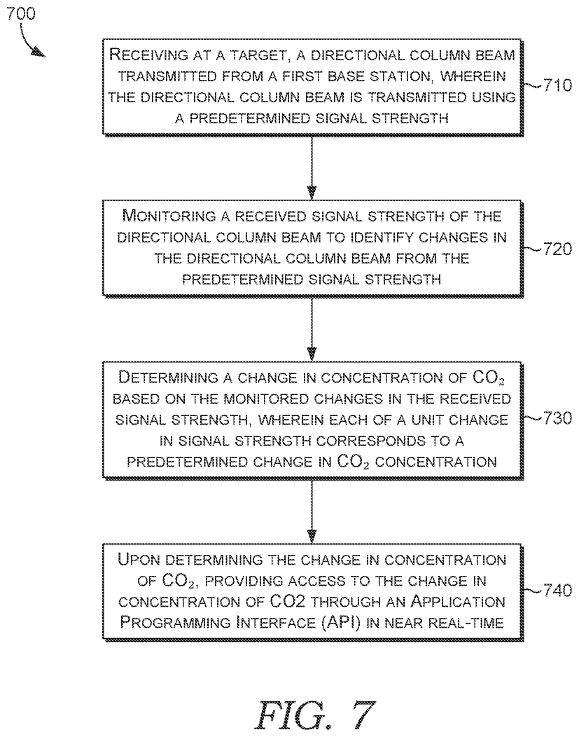

Turning now to FIG. 7, a flow chart representing method 700 is provided. In the initial stage of method 700, as illustrated in block 710, a target apparatus or entity receives an RF signal transmitted from a first base station at a predetermined signal strength. The predetermined signal strength serves as a standard parameter, facilitating a controlled environment where variations can be identified and analyzed. Transitioning to block 720, the method involves monitoring the received signal strength of the RF signal, identifying any deviations from the initially established predetermined signal strength. Following the monitoring step, block 730 provides for a process that determines greenhouse gas concentrations based on the monitored changes in the received signal strength. Each unit change in signal strength is correlated to a predetermined change in greenhouse gasses concentration. Continuing at block 740, upon discerning changes in greenhouse gasses concentration, the system leverages an API to provide near real-time access to the determined changes in greenhouse gasses concentration. Additionally, an alert system may be configured to notify a user of an increase in gas concentration. The alert system may configured to generate alerts or notifications to individuals or entities in a vicinity of the target, wherein the alerts or notifications are generated when the concentration of greenhouse gasses exceeds predetermined threshold levels. In other aspects, the method can receive meteorological data from a plurality of weather stations in proximity to the plurality of target sites, and incorporate the meteorological data in the determination of changes in the concentration of the greenhouse gas

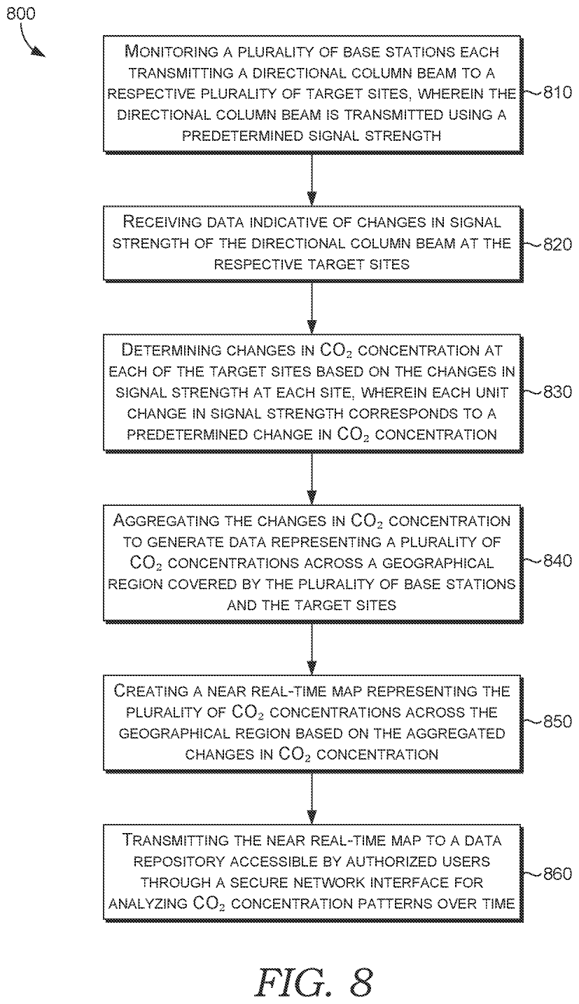

Turning now to FIG. 8, a flow chart representing method 800 is provided. In the outset of method 800, in block 810, the system engages in monitoring a multiplicity of base stations, each transmitting an RF signal targeted towards a designated set of target sites. These signals each have a predetermined signal strength. At block 820, the method requires the receipt of data indicating fluctuating signal strengths from expected signal strengths, as captured at the various target sites. In block 830, the method provides for an analysis to determine the greenhouse gasses concentration occurring at each target site. This determination is based on fluctuations in expected signal strength, translating each unit change in signal strength to a predefined change in greenhouse gasses concentration. This conversion is based on a predetermined correlation between signal strength and greenhouse gasses concentration.

At Block 840 the deduced greenhouse gasses concentrations are collated, generating a dataset that portrays the greenhouse gasses concentrations dispersed across a geographical region covered by the collective span of the base stations and target sites. At block 850, a dynamic map that offers a spatial representation of greenhouse gasses concentrations is generated based on the greenhouse gasses concentrations and the location of each cell site and target site. The method at block 860 transmits the generated near real-time map to a secure data repository. This repository is structured to allow access exclusively to authorized users. Through a secure network interface, users can access and analyze the database of greenhouse gasses concentration patterns over time, offering a tool for informed decision-making and environmental strategy formulation.

Many different arrangements of the various components depicted, as well as components not shown, are possible without departing from the scope of the claims herein. Embodiments of the technology have been described with the intent to be illustrative rather than restrictive. Alternative embodiments will become apparent to readers of this disclosure after and because of reading it. Alternative methods of implementing the aforementioned subject matter may be performed without departing from the scope of the claims herein. Certain features and sub-combinations are of utility and may be employed without reference to other features and sub-combinations, which is contemplated as within the scope of the claims.

What is claimed is:

1. A system comprising:
a target site comprising one or more processors and one or more computer storage hardware devices storing computer-usable instructions, that, when used by the one or more processors, cause the one or more processors to:
receive a signal transmitted from a base station, the signal having a predetermined signal strength, wherein the signal comprises a plurality of protocol data units encoded according to a cellular telecom protocol;
monitor changes in a detected signal strength from the predetermined signal strength of the signal;
determine changes in a gas concentration based on the monitored changes in the detected signal strength, wherein each unit of a change in the detected signal strength from an expected signal strength corresponds to a predetermined change in the gas concentration
upon determining changes in the gas concentration, providing near real-time access to the changes in the gas concentration though an application programming interface (API).

2. The system of claim 1, wherein the gas concentration is a concentration of carbon dioxide, methane, carbon monoxide, or airborne particulates.

3. The system of claim 1, wherein the one or more processors are configured to calibrate the predetermined changes in the gas concentration based on controlled gas concentrations or reference data sets.

4. The system of claim 1, wherein the target site is integrated into existing infrastructure, such as cellular towers, buildings, or transportation systems.

5. The system of claim 1, further configured to store historical data regarding gas concentrations and signal strength changes.

6. The system of claim 1, further comprising alert systems configured to notify a user of an increase in gas concentration.

7. The system of claim 1, further comprising a user interface to provide real-time visualization of the concentration of the greenhouse gas.

8. A method for detecting atmospheric greenhouse gasses utilizing a cellular network, the method comprising:
receiving at a target, a signal transmitted from a first base station, wherein the signal is transmitted using a predetermined signal strength, wherein the signal transmitted from the first base station comprises a plurality of protocol data units encoded according to a cellular telecom protocol;
monitoring a received signal strength of the signal to identify changes in the signal from the predetermined signal strength;
determining a change a concentration of a greenhouse gas based on the monitored changes in the received signal strength, wherein each of a unit change in signal strength corresponds to a predetermined change in the concentration of the greenhouse gas; and
upon determining the change in the concentration of the greenhouse gas, providing access to the change in the concentration of the greenhouse gas through an application programming interface (API) in near real-time.

9. The method of claim 8, further comprising calibrating the predetermined changes in the concentration of the greenhouse gas based on controlled experiments or reference data sets.

10. The method of claim 8, further comprising a user interface to provide real-time visualization of the concentration of the greenhouse gas.

11. The method of claim 8, wherein the greenhouse gas comprise carbon dioxide.

12. The method of claim 8, further comprising determining a change in concentration of particulate matter based on the change in signal strength.

13. The method of claim 8, further comprising alert systems configured to notify a user of an increase in gas concentration.

14. The method of claim 13, wherein the alert systems further comprise alerts or notifications to individuals or entities in a vicinity of the target, wherein the alerts or notifications are generated when the concentration of the greenhouse gas exceeds predetermined threshold levels.

15. One or more computer-readable media having computer-executable instructions embodied thereon that, when executed, perform a method comprising:
monitoring a plurality of base stations each transmitting a signal to a respective plurality of target sites, wherein the signal is transmitted using a predetermined signal strength;
receiving data indicative of changes in signal strength of the signal at the respective target sites;
determining changes in a concentration of a greenhouse gas at each of the target sites based on the changes in signal strength at each site, wherein each unit change in signal strength corresponds to a predetermined change in the concentration of the greenhouse gas;
aggregating the changes in the concentration of the greenhouse gas to generate data representing a plurality of the concentration of the greenhouse gas across a geographical region covered by the plurality of base stations and the target sites;
creating a near real-time map representing the plurality of the concentration of the greenhouse gas across the geographical region based on the aggregated changes in the concentration of the greenhouse gas; and
transmitting the near real-time map to a data repository accessible by authorized users through a secure network interface.

16. The media of claim 15, further comprising calibrating the predetermined change in the concentration of the greenhouse gas corresponding to the unit change in signal strength, wherein the calibrating utilizes historical data relating to the concentration of the greenhouse gas and signal strength changes.

17. The media of claim 15, further comprising receiving meteorological data from a plurality of weather stations in proximity to the plurality of target sites, and incorporating the meteorological data in the determination of changes in the concentration of the greenhouse gas.

18. The media of claim 15, wherein the signal is generated utilizing MMWave technology.

19. The media of claim 15, wherein each target site is either a fixed or a mobile device.

20. The media of claim 15, wherein each base station of the plurality of base stations is a part of a cellular network operating in between 30 GHz and 300 GHz.
